(19) United States
(12) Patent Application Publication
(10) Pub. No.: US 2020/0061133 A1
(43) Pub. Date: Feb. 27, 2020

(54) USE OF RECOMBINANT ONCOLYTIC TYPE II HERPES SIMPLEX VIRUS IN PREPARING MEDICINES AGAINST LYMPHOMA, ESOPHAGEAL CANCER, BREAST CANCER AND PANCREATIC CANCER

(71) Applicant: Wuhan Binhui Biotech Co., Ltd., WUHAN (CN)

(72) Inventor: Binlei Liu, WUHAN (CN)

(21) Appl. No.: 16/671,205

(22) Filed: Nov. 1, 2019

Related U.S. Application Data
(63) Continuation of application No. PCT/CN2017/091625, filed on Jul. 4, 2017.

(30) Foreign Application Priority Data
May 3, 2017 (CN) 201710304700.3

(51) Int. Cl. A61K 35/763; A61P 35/00; C12N 7/00

(52) U.S. Cl. CPC A61K 35/763; C12N 2710/16632; C12N 7/00; A61P 35/00

(57) ABSTRACT

The present invention discloses a use of recombinant oncolytic type II herpes simplex virus in preparing medicines against lymphoma, esophageal cancer, breast cancer and pancreatic cancer. The recombinant oncolytic type II herpes simplex virus can specifically grow and reproduce in human tumor cells, without affecting the proliferation of normal cells, thus it can effectively kill cancer cells, presenting good effect. These new indications provide more evidences for drug development and utilization with the recombinant oncolytic type II herpes simplex virus.

### CROSS-REFERENCE TO RELATED APPLICATIONS

The present application is a continuation-application of International Patent Application (PCT) No. PCT/CN2017/091625 filed on Jul. 4, 2017, which claims foreign priority of Chinese Patent Application No. 201710304700.3, filed on May 3, 2017, the entire contents of which are hereby incorporated by reference.

### TECHNICAL FIELD

The present invention relates to the field of biomedicine, in particular to use of recombinant oncolytic type II herpes simplex virus in preparing medicines against lymphoma, esophageal cancer, breast cancer and pancreatic cancer.

### BACKGROUND ART

Herps simplex virus (HSV) is a double-stranded DNA virus of about 154 kb in length that can replicate in the nucleus of infected host cells. HSV vectors have the following advantages: 1) a wide range of host cells; 2) a high viral titer; 3) a large capacity of exogenous genes. A disadvantage of the HSV vector is its toxicity.

A patent titled “a recombinant oncolytic type II herpes simplex virus, its preparation method and use and a tumor diagnosis kit” is disclosed in the prior art. According to the patent, a type II herpes simplex virus is disclosed, whose Latin name is Herpes Simplex Virus Type 2, deposited in China General Microbiological Culture Collection Center (CGMCC) located at No. 3, Courtyard 1, West Beichen Road, Chaoyang District, Beijing on Feb. 3, 2010, with a preservation number CGMCC No. 3600. The preserved biological material is strain H2d3d4-hGF, and the strain number has the following meaning: H2 represents type II herpes simplex virus HG52 strain (oHSV2); d3 represents deletion of ICP34.5; d4 represents deletion of ICP47; hGF represents insertion of expression kit of human granulocyte-macrophage colony stimulating factor (hGM-CSF). This patent document discloses that this recombinant oncolytic type II herpes simplex virus can be used in vivo oncolytic treatment of a variety of tumors.

### SUMMARY

In the present invention, based on continuous studies on the above virus, the inventor discovers the new uses of this virus and provides the application of the recombinant oncolytic type II herpes simplex virus in preparing medicines against lymphoma, esophageal cancer, breast cancer and pancreatic cancer.

The recombinant oncolytic type II herpes simplex virus provided in the present invention is a recombinant oncolytic type II herpes simplex virus (oHSV2) which deletes genes ICP34.5 and ICP47 of the wild type II herpes simplex virus HG52 strain. The virus strain has been deposited in China General Microbiological Culture Collection Center (CGMCC), with a preservation number CGMCC No. 3600.

The preparation process of recombinant oncolytic type II herpes simplex virus (oHSV2) and possible pharmaceutical dosage forms of the present invention have been disclosed in a Chinese patent titled “recombinant type II herpes simplex virus vector, its preparation method, recombinant virus, pharmaceutical composition and uses” (publication number: CN 102146418 B), and are not described herein again.

In the present invention, it has been confirmed through studies and experiments that oHSV2 virus strain has prominent oncolytic effect against lymphoma, esophageal cancer, breast cancer and pancreatic cancer, suggesting that the oHSV2 virus or oHSV2 viral vector has an application prospect in preparing medicines against lymphoma, esophageal cancer, breast cancer and pancreatic cancer.

### Beneficial Effects

The recombinant oncolytic type II herpes simplex virus of the present invention has been discovered to have new biological functions and it can be used for preparing medicines against lymphoma, esophageal cancer, breast cancer and pancreatic cancer. The recombinant oncolytic type II herpes simplex virus can specifically grow and reproduce in human tumor cells, without affecting the proliferation of normal cells, thus it can effectively kill cancer cells, presenting good effect. These new indications provide more evidences for drug development and utilization with the recombinant oncolytic type II herpes simplex virus.

### DESCRIPTION OF THE DRAWINGS

FIG. 1 is a diagram showing changes in tumor diameter of the right tumor after injection of oHSV2 in tumor-bearing mice of A20 mouse lymphoma models.

FIG. 2 is a diagram showing the change in tumor diameter of the left tumor after injection of oHSV2 in tumor-bearing mice of A20 mouse lymphoma models.

FIG. 3 is a diagram showing changes in tumor diameter after injection of oHSV2 in tumor-bearing mice of 4T1 mouse breast cancer models.

FIG. 4 is a diagram showing changes in tumor diameter after injection of oHSV2 in tumor-bearing nude mice of MDA-MB-231 human breast cancer models.

FIG. 5 is a diagram showing comparison of tumor diameters after injection of oHSV2 in tumor-bearing SCID mice of EC 109 esophageal cancer models.

FIG. 6 is a diagram showing changes in tumor diameter after injection of oHSV2 in tumor-bearing nude mice of MiaPaCa-2 human pancreatic cancer models.

FIG. 7 is a diagram showing changes in tumor diameter after injection of oHSV2 in tumor-bearing mice of B16 mouse melanoma models.

FIG. 8 is a diagram showing changes in tumor diameter after injection of oHSV2 in tumor-bearing nude mice of SNK-6 human NKT lymphoma models.

FIG. 9 is a diagram showing comparison of virus titers after oHSV2 infection of four tumor cells.

### DETAILED DESCRIPTION OF THE EMBODIMENTS

The invention is further described in detail below in conjunction with specific embodiments. The methods used in the following embodiments are conventional methods unless otherwise specified, and the primer synthesis and sequencing are carried out by Shanghai Sangon Biotech.

### Embodiment 1 Antitumor Effect of Recombinant Oncolytic Type II Herpes Simplex Virus (oHSV2) Against Lymphoma

A20 mouse B cell lymphoma cells were injected subcutaneously into the bilateral flanks at a dose of 2×10⁶, to induce 20 female BALB/c mice (10 animals per group) to produce tumors. The schedule for three times of injection of virus in the right flank tumor was shown in the table below.

TABLE 1
Schedule of injecting virus into A20 tumor-bearing mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

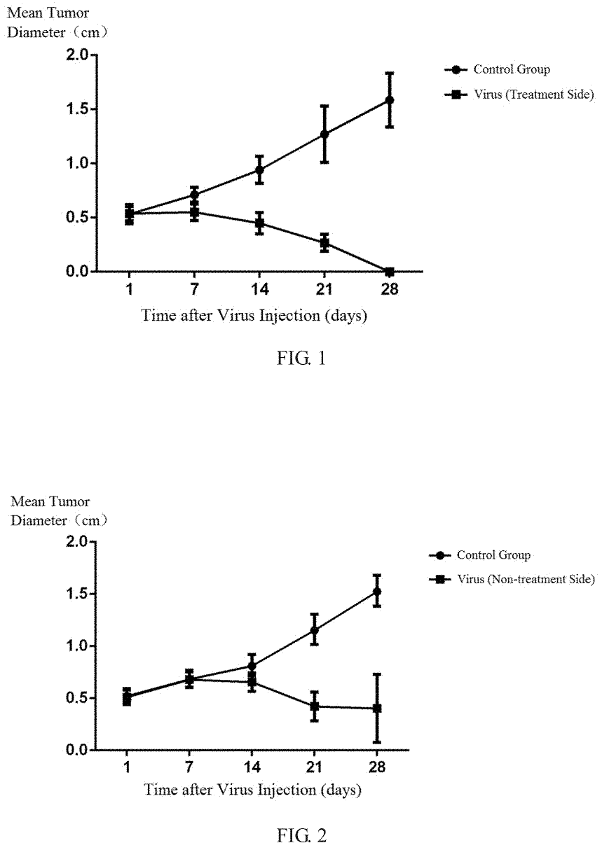

As shown in FIG. 1, after three times of injections of the virus in the right flank tumor, the tumor in the treatment group was significantly smaller than that in the control group. On the 28^{th} day after the first injection of the virus, the mean tumor diameters of the control group and the virus treatment group were 1.59 cm and 0 cm, respectively. There was significant difference in tumor diameter between the treatment group and the control group (p<0.01).

Similarly, as shown in FIG. 2, the virus also showed a good anti-tumor effect on the tumor on the non-treatment side (left flank). On the 28^{th} day after the first injection of the virus, the mean tumor diameters of the control group and the virus treatment group were 1.54 cm and 0.4 cm, respectively. The diameter of the left tumor in the treatment group was significantly different from that in the control group (p<0.05).

### Embodiment 2 Antitumor Effect of Recombinant Oncolytic Type II Herpes Simplex Virus Against Breast Cancer

(1) Antitumor effect of recombinant oncolytic type II herpes simplex virus (oHSV2) against mouse breast cancer (4T1)

4T1 mouse breast cancer cells were injected subcutaneously into the right flank at a dose of 1×10⁶, to induce 20 female BALB/c mice (10 animals per group) to produce tumors. The schedule for three times of injection of virus in the tumor was shown in the table below.

TABLE 2
Schedule of injecting virus into 4T1 tumor-bearing mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

After three times of injections of the virus in the tumor, the tumor size of the virus treatment group was significantly smaller than that in the control group. On the 28^{th} day after the first injection of the virus, the mean tumor diameters of the control group and the virus treatment group were 1.83 cm and 0.87 cm, respectively. There was significant difference in tumor diameter between the treatment group and the control group (p<0.05). The results were shown in FIG. 3.

(2) Antitumor effect of recombinant oncolytic type II herpes simplex virus (oHSV2) against human breast cancer (MDA-MB-231)

MDA-MB-231 human breast cancer cells were injected subcutaneously into the right flank at a dose of 1×10⁶, to induce 20 female BALB/c nude mice (10 animals per group) to produce tumors. The schedule for three times of injection of virus in the tumor was shown in the table below.

TABLE 3
Schedule of injecting virus into MDA-MB-231 tumor-bearing nude mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

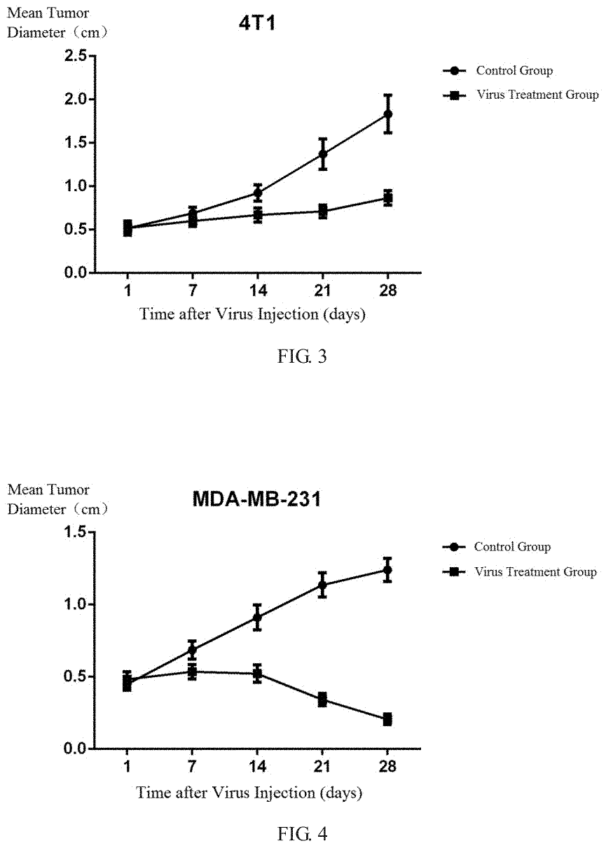

After three times of injections of the virus in the tumor, the tumor size of the virus treatment group was significantly smaller than that in the control group. On the 28^{th} day after the first injection of the virus, the mean tumor diameters of the control group and the virus treatment group were 1.24 cm and 0.21 cm, respectively. There was significant difference in tumor diameter between the treatment group and the control group (p<0.01). The results were shown in FIG. 4.

### Embodiment 3 Antitumor Effect of Recombinant Oncolytic Type II Herpes Simplex Virus (oHSV2) Against Esophageal Cancer

EC109 human esophageal cancer cells were injected subcutaneously into the right flank at a dose of 1×10⁶, to induce 20 female SCID mice (10 animals per group) to produce tumors. The schedule of three injections of virus in the tumor was shown in the table below.

TABLE 4
Schedule of injecting virus into EC 109 tumor-bearing SCID mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

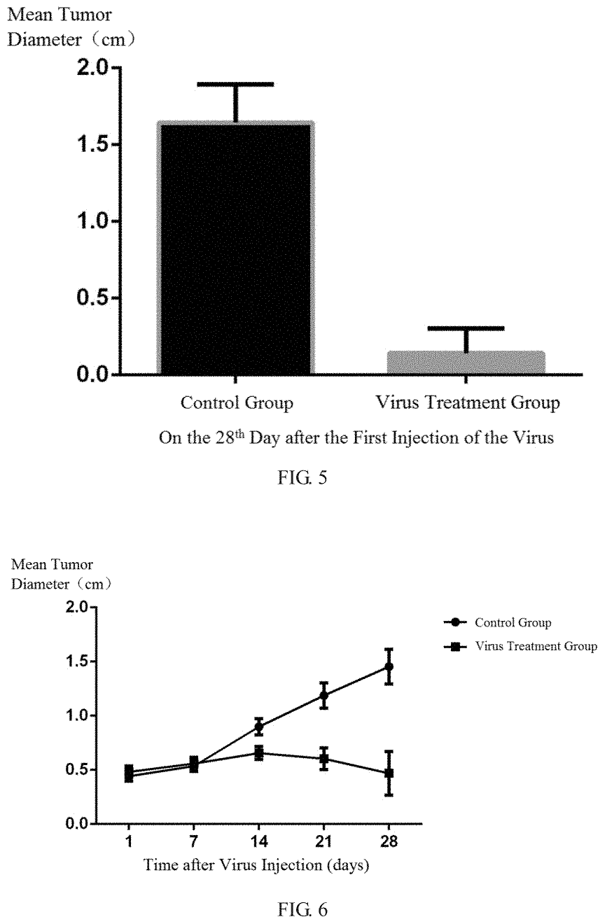

After three times of injections of the virus in the tumor, the tumor size of the virus treatment group was significantly smaller than that in the control group. On the 28^{th} day after the first injection of the virus, the mean tumor diameters of the control group and the virus treatment group were 1.64 cm and 0.14 cm, respectively. There was significant difference in tumor diameter between the treatment group and the control group (p<0.01). The results were shown in FIG. 5.

### Embodiment 4 Antitumor Effect of Recombinant Oncolytic Type II Herpes Simplex Virus (oHSV2) Against Pancreatic Cancer

MiaPaCa-2 human pancreatic cancer cells were injected subcutaneously into the right flank at a dose of 1×10⁶, to induce 20 female nude mice (10 animals per group) to produce tumors. The schedule for three times of injection of virus in the tumor was shown in the table below.

TABLE 5
Schedule of injecting virus into MiaPaCa-2 tumor-bearing nude mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

After three times of injections of the virus in the tumor, the tumor size of the virus treatment group was significantly smaller than that in the control group. On the 28^{th} day after the first injection of the virus, the mean tumor diameters of the control group and the virus treatment group were 1.45 cm and 0.47 cm, respectively. There was significant difference in tumor diameter between the treatment group and the control group (p<0.05). The results were shown in FIG. 6.

### Comparative Example 1 Antitumor Effect of Recombinant Oncolytic Type II Herpes Simplex Virus (oHSV2) Against Mouse B16 Melanoma (B16)

B16 melanoma cells were injected subcutaneously into the right flank at a dose of 2×10⁵, to induce 20 female C57/BL mice (10 animals per group) to produce tumors. The schedule for three times of injection of virus in the tumor was shown in the table below.

TABLE 6
Schedule of injecting virus into B16 tumor-bearing mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

After three times of injections of the virus in the tumor, the tumor size of the virus treatment group was not significantly smaller than that in the control group. On the 28^{th} day after the first injection of the virus, the mean tumor diameters of the control group and the virus treatment group were 1.44 cm and 1.50 cm, respectively. There was no significant difference in tumor diameter between the treatment group and the control group. As shown in FIG. 7, the recombinant oncolytic type II herpes simplex virus (oHSV2) had no significant anti-tumor effect on mouse B16 melanoma (B16).

### Comparative Example 2 Antitumor Effect of Recombinant Oncolytic Type II Herpes Simplex Virus (oHSV2) Against Human NKT Lymphoma (SNK-6)

SNK-6 human NKT lymphoma cells were injected subcutaneously into the right flank at a dose of 2×10⁶, to induce 20 female BALB/c nude mice (10 animals per group) to produce tumors. The schedule for three times of injection of virus in the tumor was shown in the table below.

TABLE 7
Schedule of injecting virus into SNK-6 tumor-bearing nude mice

| | | | Virus | Virus | Virus |
|---|---|---|---|---|---|
| | Number of | | Titer | Dosage | Injection |
| Group | Animals per | | (CCID50/ | (CCID50/ | Time |
| No. | Group | Virus | ml) | animal) | (days) |
| 1 | 10 | oHSV2 | 2 × 10⁷ | 1 × 10⁶ | 1, 4, 7 |
| 2 | 10 | Control | — | — | 1, 4, 7 |
| | | (serum-free | | | |
| | | medium) | | | |

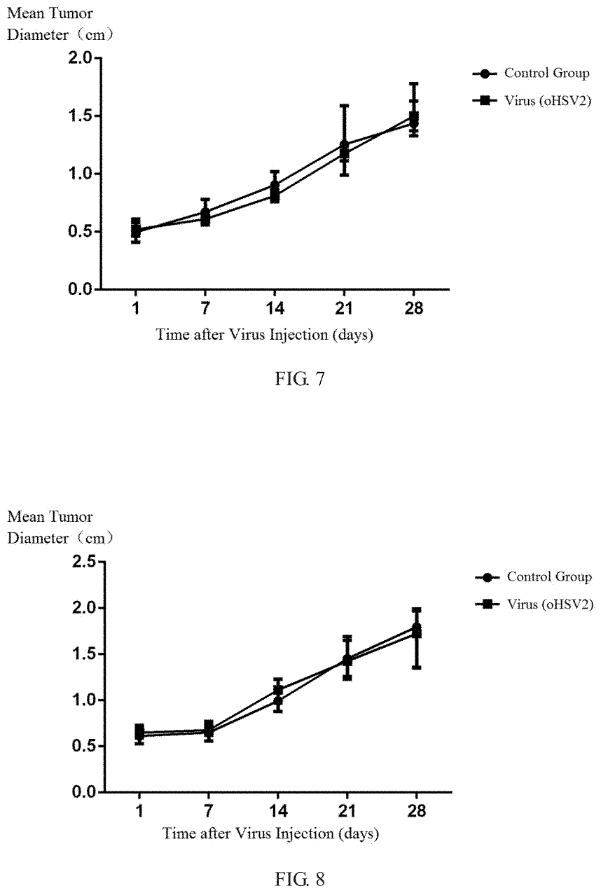

After three times of injections of the virus in the tumor, the tumor size of the virus treatment group was not significantly smaller than that in the control group. On the 28^{th} day after the first injection of the virus, the mean tumor diameters of the control group and the virus treatment group were 1.80 cm and 1.72 cm, respectively. There was no significant difference in tumor diameter between the treatment group and the control group. As shown in FIG. 8, the recombinant oncolytic type II herpes simplex virus (oHSV2) had no significant anti-tumor effect on human NKT lymphoma (SNK-6).

### Experimental Example 1 Growth Curve of Recombinant Oncolytic Type II Herpes Simplex Virus (oHSV2) in Four Cell Lines

The cells used in the experiment included the following four types: Vero cells-African green monkey kidney cells, B16 cells-mouse melanoma cells, SNK-6 cells-human NKT lymphoma cells, and CT26 cells-mouse colon cancer cells.

Experimental procedure: Four kinds of cells were paved on culture plates. When the cell confluence reached 90% or more, cells were infected with oHSV2 virus at MOI=0.01, and the virus titers were detected at the times points of 0 h, 4 h, 8 h, 16 h, 24 h, 48 h and 72 h respectively.

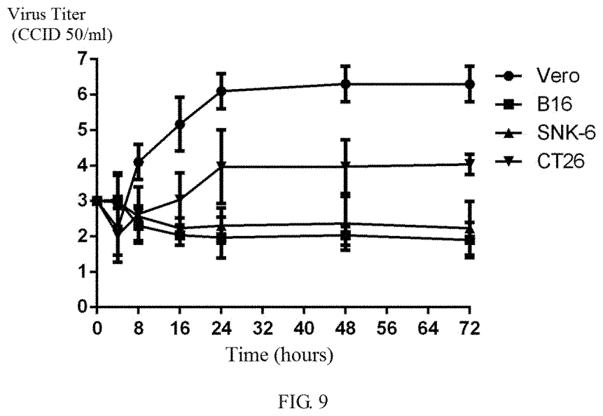

As shown in FIG. 9, the virus proliferated significantly in Vero cells, and the virus titer began to rise rapidly at 8 hours after infection, slowly rose after 24 hours, and reached the peak at 48 hours (1 g 6.3 CCID50/ml). The virus proliferated in CT-26 cells, and the virus titer reached 1 g 3.97 CCID50/ml after 24 hours and maintained until 72 hours. The viruses did not proliferate in B16 cells and SNK-6 cells.

Result Analysis:

Vero cells: African green monkey kidney cells, herpes simplex virus (HSV) sensitive cells, after infected in Vero cells, HSV proliferated to achieve a high titer.

B16 cells: Mouse melanoma cells, HSV non-sensitive cells, did not support HSV replication, so oHSV2 could not kill B16 cells.

SNK-6 cells: Human NKT lymphoma cells, HSV non-sensitive cells, did not support HSV replication, so oHSV2 could not kill SNK-6 cells.

CT26 cells: Mouse colon cancer cells, HSV semi-sensitive cells, HSV proliferated in this cell, but its titer was low.

### CONCLUSION

Since B16 and SNK-6 cells lack viral receptors, oHSV2 virus cannot proliferate in both cells. Therefore, the recombinant oncolytic type II herpes simplex virus (oHSV2) does not produce an oncolytic and anti-tumor effect on all tumors.

What is claimed is:

1. A method for treating Lymphoma, comprising administering an effective amount of a recombinant oncolytic type II herpes simplex virus, which deletes genes ICP34.5 and ICP47 of the wild type II herpes simplex virus HG52 strain.

2. A method for treating esophageal cancer, comprising administering an effective amount of a recombinant oncolytic type II herpes simplex virus, which deletes genes ICP34.5 and ICP47 of the wild type II herpes simplex virus HG52 strain.

3. A method for treating breast cancer, comprising administering an effective amount of a recombinant oncolytic type II herpes simplex virus, which deletes genes ICP34.5 and ICP47 of the wild type II herpes simplex virus HG52 strain.

4. A method for treating pancreatic cancer, comprising administering an effective amount of a recombinant oncolytic type II herpes simplex virus, which deletes genes ICP34.5 and ICP47 of the wild type II herpes simplex virus HG52 strain.
